SEQUENCE LISTING (1 sequence)

SEQ ID NO: 1 - DNA, 978 bp, Homo sapiens
cctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagagga

(19) United States
(12) Patent Application Publication
(10) Pub. No.: US 2010/0035973 A1
(43) Pub. Date: Feb. 11, 2010

(54) DISRUPTION OF PROGRAMMED DEATH 1 (PD-1) LIGAND TO ADJUVANT ADENO-ASSOCIATED VIRUS VECTOR VACCINES

(75) Inventor: Christopher Walker, Blacklick, OH (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: Nationwide Children's Hospital, Inc., Columbus, OH (US)

(21) Appl. No.: 12/373,880

(22) PCT Filed: Jul. 13, 2007
(86) PCT No.: PCT/US2007/073474

§ 371 Date: Sep. 8, 2009

Related U.S. Application Data
(60) Provisional application No. 60/831,548, filed on Jul. 17, 2006.

(51) Int. Cl. A61K 48/00; C12N 15/861; C12N 5/10

(52) U.S. Cl. 514/44 R; 435/320.1; 435/325; 435/455

(57) ABSTRACT

The invention provides for methods of modulating an immune response against a therapeutic polypeptide or an antigenic polypeptide delivered via rAAV comprising administering a modulator of programmed death-1 (PD-1) signaling.

This application claims priority benefit of U.S. Provisional Patent Application No. 601831,548, filed Jul. 17, 2006, which is incorporated by reference herein in its entirety.

### FIELD OF INVENTION

The invention provides for methods of modulating an immune response against a therapeutic polypeptide or an antigenic polypeptide comprising administering a recombinant AAV vector comprising a nucleic acid encoding a modulator of programmed death-1 (PD-1) signaling.

### BACKGROUND

Delivery of genes to humans using recombinant adeno-associated virus (rAAV) vectors is considered a promising approach for vaccination against infectious agents or treatment of inborn genetic errors. For vaccination, expression of genes derived from infectious agents (examples include the human immunodeficiency virus or the hepatitis C virus) may provide protection from infection or disease. As a therapy to correct genetic errors, rAAV-mediated delivery of a “normal” human gene to replace one that is absent or defective has the potential to reverse serious diseases (for instance dystrophin for muscular dystrophy, Factor IX for hemophilia, or CFTR for cystic fibrosis).

The quality of host cellular immunity directed against these proteins is a key factor in the success of vaccination or gene therapy using rAAV vectors. Genetic vaccination against infectious agents will only be effective if encoded viral or microbial proteins elicit functional humoral and/or cellular immunity. On the other hand prolonged expression of a therapeutic protein can only be achieved if host immunity is avoided or dampened.

There is a need for methods of enhancing host cellular immunity against vaccine proteins or suppressing these responses against therapeutic proteins that are encoded from genes delivered by rAAV vectors. One mechanism for regulating cellular immunity to vaccine or therapeutic proteins is to control expression of (i) CD28-related inhibitory molecules on T lymphocytes and/or (ii) their ligands on the target cells transduced by rAAV vectors. Examples of CD28-related inhibitory molecules expressed by T lymphocytes include CTLA-4 (cytotoxic T lymphocyte antigen 4), BTLA (B and T lymphocyte attenuator) and PD-1 (programmed death 1). Ligands for these inhibitory molecules include CD80 and CD86 for CTLA-4, PD-L1 (B7-H1) and PD-L2 (B7-DC) for PD-1 and the herpes virus entry mediator (HVEM) for BTLA.

PD-1 is a 55 kDa type 1 transmembrane protein that is a member of the CD28 family and negatively regulates immune responses. The negative regulation of the immune functions by PD-1 is thought to play a role in peripheral self tolerance, prevention of tissue destruction and protection against autoimmunity. PD-1 is induced on T cells, B cells and myeloid cells in vitro and is predominantly expressed on activated T cells in vivo. PD-L1 (B7-H1) and PD-L2 (B7-DC) are ligands for PD-1. Ligand binding to PD-1 leads to recruitment of SHP-2 and thereby inhibits antigen receptor-mediated signaling. (Okazaki et al., Curr. Opin. Immunol. 14: 779-782, 2002).

### SUMMARY OF INVENTION

The interaction between CD28-related co-inhibitory molecules on T lymphocytes and their appropriate ligand on target cells can deliver a negative or suppressive signal that impairs immune effector functions including cell-mediated cytotoxicity and/or production of cytokines such as interferon-gamma.

In animal models of gene therapy, the rAAV vector is known to induce long-term expression of the therapeutic protein. However, in human rAAV vector expression of the therapeutic polypeptide has been shown to be short lived. For example, Manno et al. (Nat. Med. 12: 342-346, 2006) demonstrated that liver tissue can be transduced with rAAV-2 expressing Factor IX to treat hemophila without acute toxicity. However, expression of Factor IX gradually declined about 5 weeks after transduction. T cell-mediated immunity to rAAV capsid antigen was suggested to destroy rAAV-2 transduced hepatocytes. It is well known that most humans are naturally infected with rAAV-2 during childhood and therefore most humans would have natural T cell immunity to rAAV-2. A role for immunity against the encoded therapeutic gene (in this example Factor IX) also cannot be formally excluded. As rAAV is an attractive vector for gene therapy in humans, it would be advantageous to suppress T-cell mediated immunity to the rAAV-transduced cells. In addition, suppression of T-cell immunity to AAV capsid proteins and the encoded therapeutic protein expressed in rAAV-transduced cells will also prolong expression of the therapeutic polypeptide which is not as long-lasting in humans when compared to animal studies.

The fact that AAV-transduced cells can express therapeutic protein encoded by the delivered gene for a period of time without being destroyed by the host immune response indicates some level of immune suppression does exist. The observations described in Examples 1 and 2 herein demonstrate that, among the many potential molecules that modulate the immune response, it is primarily the PD-L1 expression on rAAV-transduced cells that provides protection from immune elimination by CD8+ T cells that express PD-1.

The invention provides for methods of stimulating expression of PD-L1 in rAAV-transduced cells for gene therapy applications and to reduce or eliminate it for vaccine usage where PD-L1 causes T cells to lose immune function. The invention thus provides for methods of modifying the genetic payload of rAAV vectors to (i) interrupt this PD-1:PD-L1 interaction where more effective priming of immunity by vaccine antigens is required, or (ii) to stimulate this interaction to promote long-term avoidance of cellular immunity when gene therapy is the goal. In particular, the methods and vectors of the invention apply to altering the interaction of PD-1 positive T lymphocytes with its PD-L1 ligand on target cells to improve gene therapy or vaccination applications of rAAV vectors.

The invention provides for method of modulating an immune response in a mammal treated with a recombinant AAV vector comprising administering to said mammal a first nucleic acid encoding an antigenic or therapeutic polypeptide and a second nucleic acid encoding a modulator of PD-1 signaling. One or both of the nucleic acids are delivered via a recombinant AAV vector. Administration of the first nucleic acid and the second nucleic acid to the mammal may be in the same recombinant AAV vector or in two different recombinant AAV vectors. Where two separate recombinant AAV vectors are utilized, administration may be concurrent or sequential, and the rAAV vectors may be in the same composition or in different compositions. Thus the methods of modulating an immune response include administering a single recombinant AAV vector comprising the first and second nucleic acids. In addition, the method of modulating an immune response includes administering two recombinant AAV vector wherein a first recombinant AAV vector comprises the first nucleic acid and a second recombinant AAV vector comprises the second nucleic acid.

The term “PD-1 signaling” refers to activation of the PD-1 receptor, including ligand binding, and the resulting downstream signaling cascade that results in suppression of immune responses. PD-1 is a transmembrane receptor that is activated upon binding to one of two ligands PD-L1 or PD-L2. Upon ligand binding, PD-1 recruits SHP-2 and thereby inhibits T cell immune responses.

Modulators of PD-1 signaling either induce signaling through PD-1 or inhibit signaling through PD-1. Enhancers of PD-1 signaling stimulate, promote or increase PD-1 signaling by increasing or stimulating PD-L1 activity, increasing or stimulating PD-1 expression, increasing or stimulating PD-L2 activity, increasing or stimulating PD-L2 expression or inducing, and promoting interaction between PD-1 and its ligands, such as PD-L1 or PD-L2. Enhancers of PD-1 signaling includes the PD-L2 polypeptide and perhaps the PD-L1 polypeptide. Inhibitors of PD-1 signaling reduce or suppress PD-1 signaling by suppressing, suppressing or decreasing PD-1 activity, suppressing or decreasing PD-1 expression, suppressing or decreasing activity of PD-1 ligands. e.g. PD-L1 or PD-L2, inhibiting or decreasing expression of PD-1 ligands, e.g. PD-L1 or PD-L2, or suppressing the interaction of PD-1 and its ligands, e.g. PD-L1 or PD-L2. Specific examples of agents that are enhancers of PD-1 signaling and agents that are inhibitors of PD-1 signaling are described in further detail below.

The term “modulating an immune response” includes methods of negatively modulating an immune response and methods of positively modulating an immune response. Negative modulation includes inhibiting, reducing or suppressing a cellular or humoral immune response. Negative modulators of immune responses include enhancers of PD-1 signaling. Positive modulation includes inducing, stimulating or enhancing a cellular or humoral immune response. Positive modulators of immune responses include inhibitors of PD-1 signaling. Positive modulation of an immune response can enhance an existing immune response or elicit an initial immune response. For example, enhancing an immune response through inhibition of PD-1 signaling is useful in cases of infections with microbes, e.g., bacteria, viruses, fungi or parasites, or in cases of immunosuppression.

In one embodiment, the invention provides for methods of modulating an immune response in a mammal treated with a recombinant AAV vector comprising administering to said mammal a first nucleic acid encoding an antigenic polypeptide and a second nucleic acid encoding an inhibitor of PD-1 signaling, optionally in the same or a different recombinant AAV vector. The invention provides methods wherein the second nucleic acid is administered in an amount effective to inhibit PD-1 signaling and the first nucleic acid is administered in an amount effective to elicit an immune response in said mammal to said antigenic polypeptide. Preferably, the immune response elicited by the antigenic polypeptide is enhanced compared to the immune response in the absence of inhibition of PD-1 signaling.

The terms “antigenic polypeptide” and “antigen” refers to any full length polypeptide, truncation of a polypeptide or a fragment of a polypeptide that is capable of eliciting an immune response in a mammal. Antigenic polypeptides and antigen also include small immunogenic peptides. An antigenic polypeptide or antigen refers to a molecule containing one or more epitopes (either linear, conformational or both) or immunogenic determinants that will stimulate a host's immune-system, such as a mammal's immune system, to make a humoral and/or cellular antigen-specific response. The terms “antigenic polypeptide” and “antigen” and “immunogen” are used interchangeably. In one embodiment, the antigenic polypeptide is a polypeptide obtained from a microbe such as a bacteria, virus, parasite or fungus and the immune response is protective. In another embodiment, the antigenic polypeptide is a cancer-associated polypeptide and the immune response is therapeutic.

An antigen may be a whole protein, a truncated protein, a fragment of a protein or a peptide. Antigens may be naturally occurring, genetically engineered variants of the protein, and nucleic acids encoding the antigens may be codon optimized for expression in a particular mammalian subject or host. Thus, for purposes of the present invention, an “antigen” refers to a protein which includes modifications, such as deletions, additions and substitutions, generally conservative in nature, to the naturally occurring sequence, so long as the protein maintains the ability to elicit an immunological response, as defined herein. These modifications may be deliberate, as through site-directed mutagenesis, or may be accidental, such as through mutations of hosts which produce the antigens. Generally, a B-cell epitope will include at least about 5 amino acids but can be as small as 3-4 amino acids. A T-cell epitope, such as a CTL epitope, will be at least about 7-9 amino acids, and a helper T-cell epitope at least about 12-20 amino acids.

Normally, an epitope targeted by antibodies will include between about 7 and 15 amino acids, such as, 9, 10, 12 or 15 amino acids. Antibodies such as anti-idiotype antibodies, or fragments thereof, and synthetic peptide mimotopes, that is synthetic peptides which can mimic an antigen or antigenic determinant, are also captured under the definition of antigen as used herein.

An “immunological response” or “immune response” to an antigen, or vector or vaccine or composition comprising the antigen, is the development in a mammalian subject of a humoral and/or a cellular immune response to an antigen or antigens present in a vector set. For purposes of the present invention, a “humoral immune response” refers to an immune response mediated by antibody molecules or immunoglobulins. Antibody molecules of the present invention include the classes of IgG (as well as subtypes IgG 1, IgG 2a, and IgG2b), IgM, IgA, IgD, and IgE. Antibodies functionally include antibodies of primary immune response as well as memory antibody responses or serum neutralizing antibodies. The antibodies of the present invention also include cross-reactive, cross protective or cross clade antibody responses. Antibodies of the invention may serve to, but are not required to, neutralize or reduce infectivity and/or mediate antibody-complement or antibody-dependent cell cytotoxicity (ADCC) to the antigen of the pathogen.

In another embodiment, the invention provides for methods of modulating an immune response in a mammal treated with a recombinant AAV vector comprising administering to said mammal a first nucleic acid that encodes a therapeutic polypeptide and a second nucleic acid that encodes an enhancer of PD-1 signaling, or optionally in the same or a different recombinant AAV vector.

The invention provides methods wherein the second nucleic acid is administered in an amount effective to enhance PD-1 signaling and the first nucleic acid is administered in an amount effective to produce a therapeutic response in said mammal to said therapeutic polypeptide. Preferably, the enhancer of PD-1 signaling provides prolonged reduction or avoidance of an immune response to the therapeutic polypeptide such that the suppression of immune response is increased and/or prolonged compared to immune response in the absence of enhanced PD-1 signaling. The invention provides methods wherein the enhancer of PD-1 signaling is a transcriptional regulatory element targeted to upregulate endogenous PD-L1 expression or wherein the enhancer is PD-L1 polypeptide or fragment thereof Exemplary transcriptional regulatory elements include endogenous and heterologous promoters and enhancers.

The term “therapeutic polypeptide” refers to any polypeptide or fragment thereof administered to correct a physiological defect including inborn genetic errors, to replace a protein that is not expressed or expressed at low level in a subject or to alleviate, prevent or eliminate a disease state or condition in a subject. The term “therapeutic efficacy” refers to ability to of the therapeutic polypeptide to (a) prevent the development of a disease state or pathological condition, either by reducing the likelihood of or delaying onset of the disease state or pathological condition or (b) reduce or eliminate some or all of the clinical symptoms associated with the disease state or pathological condition. Examples of therapeutic polypeptides are dystrophin for treating muscular dystrophy, Factor IX for treating hemophilia, or CFTR for treating cystic fibrosis.

An embodiment of the invention provides for methods of inhibiting an immune response to a therapeutic polypeptide comprising administering a rAAV vector containing nucleic acids encoding both the therapeutic polypeptide and PD-L1, wherein PD-L1 is delivered in an amount and for a length of time effective to inhibit the immune response and/or prolong inhibition of the immune response naturally observed with rAAV vector administration. Alternatively the nucleic acid encoding the therapeutic polypeptide and nucleic acid encoding PD-L1 may be delivered in different rAAV vectors, administered in the same composition, or administered different compositions either concurrently or sequentially. The invention also provides for methods of inhibiting an immune response wherein the level of antibodies to the therapeutic polypeptide is decreased relative to the level in the absence of enhanced PD-1 signaling.

The invention also provides for methods of inhibiting an immune response to a therapeutic polypeptide wherein the expression of the therapeutic polypeptide is prolonged or the therapeutic efficacy of the therapeutic polypeptide is prolonged.

The invention further provides for methods of inhibiting an immune response to a therapeutic polypeptide wherein the dose of the nucleic acid encoding the therapeutic polypeptide is reduced compared to the dose in the absence of enhanced PD-1 signaling.

In one embodiment of methods of the invention, a composition of the invention is administered as a priming dose followed by one or more booster doses. Co-administration of proteins or polypeptides that beneficially enhance the immune response such as cytokines (e.g., IL-2, IL-12, GM-CSF), cytokine-inducing molecules (e.g. Leaf) or co-stimulatory molecules is also contemplated. Similarly, co-administration of proteins or polypeptides that beneficially inhibit the immune response is also contemplated such as CD40, CD40 ligand, antibodies against cytokines or immunosuppressive drugs.

While the data described herein indicate that rAAV vectors are unique in their PD-1 signaling profile, it is contemplated that other viral vectors also utilize the PD-1 signaling pathway to cause immune suppression. Thus, the methods of the invention as described herein may be carried out, and the compositions of the invention as described herein may be made, with any gene delivery vectors based on viruses, e.g. RNA viruses or DNA viruses, including rAAV-vectors and non-AAV viral vectors, such as for example adenovirus, retrovirus, lentivirus, alphavirus, pox viruses, herpes virus, polio virus, and sindbis virus and vaccinia viruses.

In a further embodiment, the invention provides for a recombinant AAV vector comprising a first nucleic acid encoding an antigenic or therapeutic polypeptide and a second nucleic acid encoding a modulator of PD-1 signaling, and for compositions comprising such recombinant AAV vectors. The invention also provides for cells transduced with the recombinant AAV vector of the invention and immunogenic and/or vaccine compositions comprising the recombinant AAV vector of the invention.

The present invention also provides kits that comprise a vector of the present invention and may include components necessary to administer a vector of the invention and/or to carry out the methods of the invention. The kits also comprise suitable buffers and instructions for performing the methods of the present invention. Procedures using these kits can be performed by clinical laboratories, experimental laboratories, medical practitioners, or private individuals.

In another embodiment, the invention provides for methods of producing a recombinant AAV vector of the invention comprising the steps of (a) introducing an rAAV vector comprising a first nucleic acid encoding an antigenic or therapeutic polypeptide and optionally a second nucleic acid encoding a modulator of PD-1 signaling into a host cell; (b) introducing an AAV helper virus construct into the host cell; (c) introducing a helper virus into the host cell; and (d) culturing the host cell to produce recombinant AAV virions of the rAAV vector.

The invention provides for a composition comprising (a) a first recombinant AAV vector comprising a first nucleic acid encoding an antigenic or therapeutic polypeptide and (b) a second recombinant AAV vector comprising a second nucleic acid encoding a modulator of PD-1 signaling.

Inhibition of PD-1 Signaling in Cells Transduced by rAAV Vectors to Enhance Immune Responses

Eliminating or reducing expression of PD-L1 and/or PD-L2 on host cells transduced by rAAV vectors will render them susceptible to immune attack by CD8+ or CD4+ T cells. Therefore, the invention provides methods of destroying antigen-producing cells in order to create an optimal formation of immunological memory and future protection from infection. In particular exemplary embodiments, expression of PD-L1 in rAAV-transduced cells will be accomplished by constructing rAAV vectors that have two genetic elements. The first genetic element is a gene encoding a protective antigen (protein) derived from an infectious agent such as a parasite, virus, or bacterium for the purposes of eliciting protective immunity in humans. The second genetic element encodes an inhibitor of PD-1 signaling such as a silencing RNA (siRNA) that specifically targets PD-L1 RNA transcribed from the host genome for destruction. Delivery of inhibitors of PD-1 signaling may be accomplished either in the same vector as the encoded vaccine protein(s), or in a separate vector that is delivered to the same tissue site or local area and will infect the same cell or a nearby cell.

Inhibitors of PD-1 signaling are useful to enhance immunity to antigenic polypeptides. An inhibitor of PD-1 signaling can be used prophylactically in vaccines against various polypeptides, e.g., polypeptides derived from pathogens. Immunity against a pathogen, e.g., a virus, can be induced by vaccinating with a viral polypeptide along with an agent that inhibits PD-1 signaling, in an appropriate adjuvant.

Alternatively, a vector comprising genes which encode for both a antigenic polypeptide and an inhibitor of PD-1 signaling can be used for vaccination. In another embodiment, the antigenic polypeptide in the vaccine is a self-antigen. Such a vaccine is useful in the modulation of tolerance in an organism. Immunization with a self antigen and an agent that inhibitors PD-1 signaling can break tolerance (i.e., interfere with tolerance of a self antigen). Such a vaccine may also include other adjuvants such as alum or cytokines (e.g., GM-CSF, IL-12, B7-1, or B7-2).

Inhibition of PD-1 signaling is also therapeutically useful in situations where upregulation of antibody and cell-mediated responses, resulting in more rapid or thorough clearance of a virus, bacterium, or parasite, would be beneficial. These conditions include viral skin diseases such as Herpes or shingles, in which case such an agent can be delivered topically to the skin. In addition, systemic viral diseases such as influenza, the common cold, and encephalitis might be alleviated by the administration of such agents systemically. In certain instances, it may be desirable to further administer other agents that upregulate immune responses, for example, forms of B7 family members that transduce signals via costimulatory receptors, in order further augment the immune response.

Inhibitors of PD-1 signaling can be used as adjuvants to boost responses to foreign antigens in the process of active immunization. In one exemplary embodiment, immune cells are obtained from a subject and cultured ex vivo in the presence of an agent that that inhibits PD-1 signaling, then administered to a subject. Immune cells can be stimulated to proliferate in vitro by, for example, providing the immune cells with a primary activation signal and a costimulatory signal, as is known in the art.

Exemplary inhibitors of PD-1 signaling are antisense nucleic acids, triplex oligonucleotides, ribozymes, or silencing RNA which inhibit expression of PD-1 or its ligands. An oligonucleotide complementary to the area around a PD-1, PD-L1 or PD-L2 polypeptide translation initiation site can be synthesized. One or more antisense oligonucleotides can be added to cell media or administered to a patient to prevent the synthesis of the PD-1 or its ligands. The antisense oligonucleotide is taken up by cells and hybridizes to a PD-1, PD-L1 or PD-L2 mRNA to prevent translation. Alternatively, an oligonucleotide which binds double-stranded DNA to form a triplex construct to prevent DNA unwinding and transcription can be used. As a result of either, synthesis of or PD-1, PD-L1 or PD-L2 polypeptide is blocked. When PD-1 or its ligand expression is modulated, preferably, such modulation occurs by a means other than by knocking out the PD-1, PD-L1 or PD-L2 gene.

The invention also contemplates using RNA interference (RNAi) technology on molecules involved in PD-1 signaling, such as inhibiting PD-1 or PD-L1 expression. RNAi, known to occur in animals and eukaryotes, is a process in which double stranded RNA (dsRNA; typically >200 nucleotides in length) triggers the destruction of mRNAs sharing the same sequence. RNAi is initiated by the conversion of dsRNA into 21-23 nucleotide fragments and these small interfering RNAs (siRNAs) direct the degradation of target RNAs. (Elbashir et al., Nature 411, 494-498 (2001), Fire et al., Nature 391, 199-213 (1998), Hannon, G. J., Nature 418, 244-251 (2002)). It has been rapidly adopted to use for silencing genes in a variety of biological systems (Reich et al., Mol. Vis. 9, 210-216 (2003), Song et al., Nat. Med. 9, 347-351 (2003)).

RNAi technology may be carried out in mammalian cells by transfection of siRNA molecules. The siRNA molecules may be chemically synthesized, generated by in vitro transcription, or expressed by a vector or PCR product. Commercial providers such as Ambion Inc. (Austin, Tex.), Darmacon Inc. (Lafayette, Colo.), InvivoGen (San Diego, Calif.), and Molecular Research Laboratories, LLC (Herndon, Va.) generate custom siRNA molecules. In addition, commercially kits are available to produce custom siRNA molecules, such as SILENCER™ siRNA Construction Kit (Ambion Inc., Austin, Tex.) or psiRNA System (InvivoGen, San Diego, Calif.). These siRNA molecules may be introduced into cells through transient transfection or by introduction of a expression vectors that continually express the siRNA in transient or stably transfected mammalian cells. Transfection may be accomplished by well known methods including methods such as infection, calcium chloride, electroporation, microinjection, lipofection or the DEAE-dextran method or other known techniques.

The invention particularly provides for introducing the siRNA molecules into a cell in vivo by local injection of or by other appropriate viral or non-viral delivery vectors. Hefti, Neurobiology, 25:1418-1435 (1994). For example, the siRNA molecule may be contained in an rAAV vector for delivery to the targeted cells (e.g., Johnson, International Publication No. WO95/34670; International Application No. PCT/US95/07178). The recombinant AAV genome typically contains AAV inverted terminal repeats flanking the siRNA sequence operably linked to functional promoter and polyadenylation sequences. Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors.

The preferred siRNA molecule is 20-25 base pairs in length, most preferably 21-23 base pairs, and is complementary to the target gene sequence. The siRNA molecule preferably has two adenines at its 5′ end. The siRNA sequences that contain 30-50% guanine-cytosine content are known to be more effective than sequences with a higher guanine-cytosine content. Therefore, siRNA sequence with 30-50% are preferable, while sequences with 40-50% are more preferable. The preferred siRNA sequence also should not contain stretches of 4 or more threonines or adenines.

Additional exemplary agents for use in inhibiting PD-1 signaling include, e.g., combinations of antibodies that recognize and block PD-L1 and antibodies that recognize and block PD-1, and compounds that block the interaction of PD-L1 with its naturally occurring binding partner (s) on an immune cell (e.g., soluble, monovalent PD L1 molecules; soluble forms of PD-L1 that do not bind to Fc receptors on antigen presenting cells; soluble forms of PD-1; or compounds identified in the subject screening assays). The invention provides for use of soluble PD-1 fragments, including fusion proteins comprising PD-1 polypeptide fragments.

Reinforcing PD-L1 Expression in rAAV Transduced Cells to Prolong Expression of Therapeutic Genes.

PD-L1 expression in tissues is not constitutive and PD-L1 would not necessarily be present on the surface of rAAV-transduced cells indefinitely. There is evidence that its expression is increased by cytokines of the immune system, and thus may not be stable. Even transient loss of PD-L1 expression could result in an immune attack on rAAV-tranduced cells producing a therapeutic protein.

To re-enforce expression of PD-L1 on rAAV transduced cells, in one exemplary embodiment, rAAV vectors will be engineered to contain two genetic elements. The first genetic element is a nucleic acid encoding a therapeutic polypeptide. The second genetic element is a nucleic acid encoding PD-L1 polypeptide. For example, a POL I or POL II promoter is used to regulate expression of the PD-L1 gene in the rAAV vector. Alternatively, the nucleic acid encoding the therapeutic polypeptide and the nucleic acid encoding PD-L1 are encoded by separate rAAV vectors that are delivered to the same target cell or to cells nearby each other in the same tissue site or local area. Delivery of these rAAV vectors may be concurrent or sequential.

In one embodiment of the invention, tolerance is induced against specific antigens by co-administering an antigen with an enhancer of PD-1 signaling. For example, tolerance can be induced to specific polypeptides. In one embodiment, immune responses to allergens or foreign polypeptides to which an immune response is undesirable can be inhibited by enhancers of PD-1 signaling. For example, patients that receive Factor VIII frequently generate antibodies against this clotting factor. Co-administration of enhancer of PD-1 signaling, with recombinant factor VIII (or physically linking PD-L1 to Factor VIII, e.g., by cross-linking) can result in negative modulation of the immune response.

Negative modulation of immune responses by enhancing or stimulating PD-1 signaling is useful in downmodulating the immune response, e.g., in situations of tissue, skin and organ transplantation, in graft-versus-host disease (GVHD), or allergies, or in autoimmune diseases such as systemic lupus erythematosus and multiple sclerosis. For example, blockage of immune cell function results in reduced tissue destruction in tissue transplantation. Typically, in tissue transplants, rejection of the transplant is initiated through its recognition as foreign by immune cells, followed by an immune reaction that destroys the transplant. The administration of a molecule which enhances or stimulates PD-1 signaling alone or in conjunction with another immunosuppressive agent prior to or at the time of transplantation can inhibit the generation of a costimulatory signal. Moreover, enhancing or stimulating PD-1 signaling may also be sufficient to anergize the immune cells, thereby inducing tolerance in a subject. Induction of long-term tolerance by enhancing PD-1 signaling may avoid the necessity of repeated administration of these activating reagents.

To achieve sufficient immunosuppression or tolerance in a subject, it may also be desirable to block the costimulatory function of other molecules. For example, it may be desirable to block the function of B7-1 and B7-2 by administering a soluble form of a combination of peptides having an activity of each of these antigens or between CD40 and CD40 ligand (e.g., anti CD40 ligand antibodies), antibodies against cytokines, or immunosuppressive drugs.

For example, enhancing PD-1 signaling may also be useful in treating autoimmune disease. Many autoimmune disorders are the result of inappropriate activation of immune cells that are reactive against self tissue and which promote the production of cytokines and autoantibodies involved in the pathology of the diseases. Preventing the activation of autoreactive immune cells may reduce or eliminate disease symptoms. Administration of enhancers of PD-1 signaling may induce antigen-specific tolerance of autoreactive immune cells which could lead to long-term relief from the disease. Additionally, co-administration of agents which block costimulation of immune cells by disrupting receptor-ligand interactions of B7 molecules with costimulatory receptors may be useful in inhibiting immune cell activation to prevent production of autoantibodies or cytokines which may be involved in the disease process. The efficacy of reagents in preventing or alleviating autoimmune disorders can be determined using a number of well-characterized animal models of human autoimmune diseases. Examples include murine experimental autoimmune encephalitis, systemic lupus erythematosus in MRL/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, N.Y., 1989, pp. 840-856).

Inhibition of immune cell activation is useful therapeutically in the treatment of allergies and allergic reactions, e.g., by inhibiting IgE production. An agent that enhances PD-1 signaling can be administered to an allergic subject to inhibit immune cell-mediated allergic responses in the subject. Stimulation of PD-1 signaling can be accompanied by exposure to allergen in conjunction with appropriate MHC molecules. Allergic reactions can be systemic or local in nature, depending on the route of entry of the allergen and the pattern of deposition of IgE on mast cells or basophils. Thus, immune cell-mediated allergic responses can be inhibited locally or systemically by administration of an enhancer of PD-1 signaling.

### AAV Vectors

Adeno-associated virus (AAV) is a non-pathogenic parvovirus, the single-stranded DNA genome of which is about 4.7 kb in length including 145 nucleotide inverted terminal repeat (ITRs). There are at least eight recognized serotypes of human AAV, designated as AAV-1 , AAV-2, AAV-3, AAV-4, AAV-5, AAV-6, AAV-7 and AAV-8. AAV vectors may also be constructed from AAV viruses harbored by non-human primates including baboons, chimpanzees, and various species of monkeys. Examples include AAV serotypes 1 through 8 from rhesus macaques.

Although 85% of the human population is seropositive for AAV-2, the virus has never been associated with disease in humans (Berns et al. Adv. Virus Res. Adv. Virus Res.; 32143006 (1987)). Recombinant AAV (rAAV) virions are of interest as vectors for vaccine preparations and gene therapy because of their broad host range, excellent safety profile, and duration of transgene expression in infected hosts.

AAV possesses unique features that make it attractive as a vector for expressing peptides/polypeptides. AAV infection of cells in culture is noncytopathic, and natural infection of humans and other animals is silent and asymptomatic. Moreover, AAV infects many cells allowing the possibility of targeting many different tissues in vivo. AAV infects slowly dividing and non-dividing cells and can persist essentially for the lifetime of those cells as a transcriptionally active nuclear episome (extrachromosomal element) and that integrated copies of vector in organs such as liver or muscle are very rare.

The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to activate adenovirus (56° to 65° C. for several hours), making cold preservation of AAV less critical. AAV may be lyophilized. Finally, AAV-infected cells are not resistant to superinfection.

The nucleotide sequences for various AAV are provided in GenBank as shown below. The complete genome of AAV-1 is provided in GenBank Accession No. NC_{—}002077; the complete genome of AAV-2 is provided in GenBank Accession No. NC_{—}001401 and Srivastava et al., J. Virol., 45: 555-564 {1983); the complete genome of AAV-3 is provided in GenBank Accession No. NC_{—}1829; the complete genome of AAV-4 is provided in GenBank Accession No. NC_{—}001829; the AAV-5 genome is provided in GenBank Accession No. AF085716; the complete genome of AAV-6 is provided in GenBank Accession No. NC_{—}00 1862; and at least portions of AAV-7 and AAV-8 genomes are provided in GenBank Accession Nos AX753246 and AX753249, respectively.

In AAV cis-acting sequences directing viral DNA replication, encapsidation/packaging, and host cell chromosome integration are contained within the ITRs. Three AAV promoters, p5, p19, and p40 (named for their relative map locations), drive the expression of the two AAV internal open reading frames encoding rep and cap genes. The two rep promoters (p5 and p19), coupled with the differential splicing of the single AAV intron result in the production of four rep proteins (rep 78, rep 68, rep 52, and rep 40) from the rep gene. Rep proteins possess multiple enzymatic properties which are ultimately responsible for replicating the viral genome. The cap gene is expressed from the p40 promoter and it encodes the three capsid proteins VP1, VP2, and VP3. Alternative and non-consensus translational start sites are responsible for the production of the three related capsid proteins. There is a single consensus polyadenylation site located in the AAV genome. The life cycle and genetics of AAV are reviewed in Muzyczka, Current Topics in Microbiology and Immunology, 158: 97-129 (1992). When AAV infects a human cell, the viral genome may integrate into the chromosome resulting in latent infection of the cell or may persist as a stable episome. Production of infectious virus does not occur unless the cell is infected with a helper virus (for example, adenovirus or herpesvirus). In the case of adenovirus, genes EIA, E1B, E2A, E4 and VA provide helper functions. Upon infection with a helper virus, the AAV provirus is rescued and amplified, and both AAV and adenovirus are produced. Methods for purifying rAAV vectors from helper virus are known in the art and include methods disclosed in for example, U.S. Pat. No. 6,566,118 and PCT publication WO 98/09657.

rAAV vectors comprising an antigen of a pathogen can be produced by recombinant methods known to those of skill in the art. Where transcription of the heterologous polynucleotide encoding the antigen of the pathogen is desired in the intended target cell, it can be operably linked to its own or to a heterologous promoter, depending for example on the desired level and/or specificity of transcription within the target cell, as is known in the art. Various types of promoters and enhancers are suitable for use in this context. Constitutive promoters provide an ongoing level of gene transcription, and are preferred when it is desired that the therapeutic polynucleotide be expressed on an ongoing basis. Inducible promoters generally exhibit low activity in the absence of the inducer, and are up-regulated in the presence of the inducer. They may be preferred when expression is desired only at certain times or at certain locations, or when it is desirable to titrate the level of expression using an inducing agent. Promoters and enhancers may also be tissue-specific and exhibit their activity only in certain cell types, presumably due to gene regulatory elements found uniquely in those cells. Tissue specificity of the AAV infection may be directed using targeting peptides within the AAV capsid protein(s) as taught in International Patent Publication No. WO 02/053703.

Illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. Inducible promoters include heavy metal ion inducible promoters (such as the mouse mammary tumor virus (mMTV) promoter or various growth hormone promoters), and the promoters from T7 phage which are active in the presence of T7 RNA polymerase. By way of illustration, examples of tissue-specific promoters include various surfactin promoters (for expression in the lung), myosin promoters (for expression in muscle), and albumin promoters (for expression in the liver), HP1 (for expression in the liver) and PSA (for expression in the prostate). A large variety of other promoters are known and generally available in the art, and the sequences for many such promoters are available in sequence databases such as the GenBank database.

The heterologous polynucleotide may also comprise control elements that facilitate translation (such as a ribosome binding site or “RBS” and a polyadenylation signal). Accordingly, the heterologous polynucleotide will generally comprise at least one coding region operatively linked to a suitable promoter, and may also comprise, for example, an operatively linked enhancer, ribosome binding site and poly-A signal. The heterologous polynucleotide may comprise one antigen encoding region, or more than one antigen encoding region under the control of the same or different promoters. The entire unit, containing a combination of control elements and encoding region, is often referred to as an expression cassette.

The heterologous polynucleotide is integrated by recombinant techniques into or preferably in place of the AAV genomic coding region (i.e. in place of the AAV rep and cap genes), but is generally flanked on either side by AAV inverted terminal repeat (ITR) regions. This means that an ITR appears both upstream and downstream from the coding sequence, either in direct juxtaposition, preferably (although not necessarily) without any intervening sequence of AAV origin in order to reduce the likelihood of recombination that might regenerate a replication-competent AAV genome, Recent evidence suggests that a single ITR can be sufficient to carry out the functions normally associated with configurations comprising two ITRs (WO 94/13788), and vector constructs with only one ITR can thus be employed in conjunction with the packaging and production methods of the present invention.

The rAAV rep gene can also be operably linked to a heterologous promoter, whether rep is provided as part of the vector construct, or separately. Any heterologous promoter that is not strongly down-regulated by rep gene expression is suitable; but inducible promoters are preferred because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase.

Given the relative encapsidation size limits of various AAV genomes, insertion of a large heterologous polynucleotide into the genome necessitates removal of a portion of the AAV sequence. Removal of one or more AAV genes is desirable to reduce the likelihood of generating replication-competent AAV (“RCA”). Accordingly, encoding or promoter sequences for rep, cap or both may be removed, since the functions provided by these genes can be provided in trans. The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are preferably not flanked by AAV ITRs and preferably do not share any substantial homology with the rAAV genome. Thus, in order to minimize homologous recombination during replication between the vector sequence and separately provided packaging genes, it is desirable to avoid overlap of the two polynucleotide sequences. Alternatively, when insertion of a large antigen encoding polynucleotide is desired, the methods disclosed in PCT publication WO 99/60146, that describes a plurality of DNA segments, each in an individual rAAV vector, may be delivered so as to result in a single DNA molecule comprising a plurality of the DNA segments, and WO 01/25465 may be used.

AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). Transfection of such AAV recombinant plasmids into mammalian cells with an appropriate helper virus results in rescue and excision of the AAV genome free of any plasmid sequence, replication of the rescued genome and generation of progeny infectious AAV particles. Recombinant AAV vectors comprising a heterologous polynucleotide encoding an antigen of a pathogen may be constructed by substituting portions of the AAV coding sequence in bacterial plasmids with the heterologous polynucleotide. General principles of rAAV vector construction are also reviewed in for example, Carter, 1992, Current Opinions in Biotechnology, 1533-539; and Muzyczka, 1992, Curr. Topics in Microbial. and Immunol., 158:97-129). The AAV ITRs are generally retained, since packaging of the vector requires that they be present in cis. Other elements of the AAV genome, in particular, one or more of the packaging genes, may be omitted. The vector plasmid can be packaged into an AAV particle by supplying the omitted packaging genes in trans via an alternative source. In one approach, the sequence flanked by AAV ITRs (the rAAV vector sequence), and the AAV packaging genes to be provided in trans, are introduced into the host cell in separate bacterial plasmids. Examples of this approach are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828) have described a packaging plasmid called pAAV/Ad, which consists of Rep and Cap encoding regions enclosed by ITRs from adenovirus. A second approach is to provide either the vector sequence, or the AAV packaging genes, in the form of an episomal plasmid in a mammalian cell used for AAV replication. For example, U.S. Pat. No. 5,173,414 describes a cell line in which the vector sequence is present as a high-copy episomal plasmid. The cell lines can be transduced with the trans-complementing AAV functions rep and cap to generate preparations of AAV vector. A third approach is to provide either the vector sequence, or the AAV packaging genes, or both, stably integrated into the genome of the mammalian cell used for replication. One exemplary technique is outlined in international patent application WO 95/13365 (Targeted Genetics Corporation and Johns Hopkins University) and corresponding U.S. Pat. No. 5,658.776 (by Flotte et al.). This example uses a mammalian cell with at least one intact copy of a stable integrated rAAV vector, wherein the vector comprises an AAV ITR and a transcription promoter operably linked to a target polynucleotide, but wherein the expression of rep is limiting.

In a preferred embodiment, an AAV packaging plasmid comprising the rep gene operably linked to a heterologous AAV is introduced into the cell, and then the cell is incubated under conditions that allow replication and packaging of the AAV vector sequence into particles. A second exemplary technique is outlined in patent application WO 95/13392 (Trempe et al.). This example uses a stable mammalian cell line with an AAV rep gene operably linked to a heterologous promoter so as to be capable of expressing functional Rep protein. In various preferred embodiments, the AAV cap gene can be provided stably as well or can be introduced transiently (e.g. on a plasmid). A recombinant AAV vector can also be introduced stably or transiently. Another exemplary technique is outlined in patent application WO 96/17947 (by Targeted Genetics Corporation, J. Allen). This example uses a mammalian cell which comprises a stably integrated AAV cap gene, and a stably integrated AAV rep gene operably linked to a heterologous promoter and inducible by helper virus. In various preferred embodiments, a plasmid comprising the vector sequence is also introduced into the cells (either stably or transiently). The rescue of AAV vector particles is then initiated by introduction of the helper virus. Other methods for generating high-titer preparations of recombinant AAV vectors have been described. International Patent Application No. PCT/US98/18600 describes culturing a cell line which can produce rAAV vector helper virus; infecting the cells with a helper virus, such as adenovirus; and and other viral production methods and systems are also described in, for example, WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 31124-1132.

### Production of AAV Vectors

AAV vectors and virons can be produced using standard methods known to one of skill in the art. In some examples, methods for producing rAAV vectors and virions generally involve the steps of (1) introducing an AAV vector into a host cell, wherein the AAV may have certain regions/functions necessary for viral replication deleted; (2) introducing an AAV helper construct into the host cell, where the helper construct includes any necessary AAV coding regions capable of being expressed in the host cell to complement AAV viral regions/function missing from the AAV vector; (3) introducing one or more helper viruses and/or accessory function vectors as necessary into the host cell, wherein the helper virus and/or accessory function vectors provide accessory functions capable of supporting efficient recombinant AAV (rAAV) virion production in the host cell; and (4) culturing the host cell to produce rAAV virions. The AAV vector, AAV helper construct and the helper virus or accessory function vector(s) can be introduced into the host cell either simultaneously or serially, using standard transfection or transduction techniques. Additional rAAV production strategies are known in the art and are described in for example U.S. Pat. No. 5,786,211; U.S. Pat. No. 5,871,982, which discloses the use of hybrid adenovirus/AAV vectors; and U.S. Pat. No. 6,258,595, which discloses methods for helper free production of rAAV. Methods of purifying rAAV from helper virus are known in the art and disclosed in for example, U.S. Pat. No. 6,566,118 and PCT publication WO 98/09657. Production of pseudotyped rAAV is disclosed in for example PCT publication WO 01/83692. The disclosures of the foregoing patent publications are specifically incorporated herein by reference in their entirety. The rAAV vectors of the present invention are not limited in scope to any particular production or purification methods. Other methods for producing and purifying rAAV are known in the art and are encompassed within the present invention. Methods for producing non-rAAV vectors are also known in the art and are encompassed within the present invention.

### Compositions

Compositions comprising vectors of the present invention, may further comprise various excipients, adjuvants, carriers, auxiliary substances, modulating agents, and the like. An effective amount of a priming vector or boosting vector can be determined by one of skill in the art. Such an amount will fall in a range that can be determined through routine trials and are disclosed herein. A carrier, which is optionally present, is a molecule that does not itself induce the production of antibodies harmful to the individual receiving the composition. Suitable carriers are typically large, slowly metabolized macromolecules such as proteins, polysaccharides, polylactic acids, polyglycollic acids, polymeric amino acids, amino acid copolymers, lipid aggregates (such as oil droplets or liposomes), and inactive virus particles. Examples of particulate carriers include those derived from polymethyl methacrylate polymers, as well as microparticles derived from poly(lactides) and poly(lactide-coglycolides), known as PLG. See, e.g., Jeffery et al., Pharm. Res. (1993) 10:362-368; McGee J P, et al., J Microencapsul. 14(2):197-210, 1997; O'Hagan D T, et al., Vaccine 11 (2):149-54, 1993. Such carriers are well known to those of ordinary skill in the art.

Additional carriers include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Additionally, these carriers may function as immunostimulating agents (“adjuvants”). Furthermore, the antigen may be conjugated to a bacterial toxoid, such as toxoid from diphtheria, tetanus, cholera, etc., as well as toxins derived from E. coli. Such adjuvants include, but are not limited to: (1) aluminum salts (alum), such as aluminum hydroxide, aluminum phosphate, aluminum sulfate, etc.; (2) oil-in-water emulsion formulations (with or without other specific immunostimulating agents such as muramyl peptides (see below) or bacterial cell wall components), such as for example (a) MF59 (International Publication No. WO 90/114837), containing 5% Squalene, 0.5% Tween 80, and 0.5% Span 85 (optionally containing various amounts of MTP-PE (see below), although not required) formulated into submicron particles using a microfluidizer, (b) SAF, containing 10% Squalane, 0.4% Tween 80, 5% pluronic-blocked polymer L121, and MDP either microfluidized into a submicron emulsion or vortexed to generate a larger particle size ion, and (c) Ribi™ adjuvant system (RAS), (Ribi Immunochem, Hamilton, Mont.) containing 2% Squalene, 0.2% Tween 80, and one or more bacterial cell wall components from the groUP consisting of monophosphorylipid A (MPL). trehalose dimycolate (TDM), and cell wall skeleton (CWS), preferably MPL+CWS (Detoxu); (3) saponin adjuvants, such as Stimulon™. (Cambridge Bioscience, Worcester, Mass.) may be used or particle generated therefrom such as ISCOMs (immunostimulating complexes); (4) Complete Freunds Adjuvant (CFA) and Incomplete Freunds Adjuvant (IFA); (5) cytokines, such as interleukins (IL-1, IL-2, etc.), macrophage colony stimulating factor (M-CSF), tumor necrosis factor (TNF), beta chemokines (MIP, 1-alpha, 1-beta Rantes, etc.); (6) detoxified mutants of a bacterial ADP-ribosylating toxin such as a cholera toxin (CT), a pertussis toxin (PT), or an E. coli heat-labile toxin (LT), particularly LT-K63 (where lysine is substituted for the wild-type amino acid at position 63) LT-R72 (where arginine is substituted for the wild-type amino acid at position 72), CYS 109 (where serine is substituted for the wild-type amino acid at position 109), and PT-K9/G129 (where lysine is substituted for the wild-type amino acid at position 9 and glycine substituted at position 129) (see, e.g., International Publication Nos. WO 93/13202 and WO 92/19265); and (7) other substances that act as immunostimulating agents to enhance the effectiveness of the composition.

The compositions may be formulated as neutral or salt forms. Pharmaceutically acceptable salts, include the acid addition salts (formed with the free amino groups of the peptide) and which are formed with inorganic acids such as, e.g., hydrochloric or phosphoric acids, or such organic acids as acetic, oxalic, tartaric, mandelic. Salts formed with the free carboxyl groups may also be derived from inorganic bases such as, e.g., sodium, potassium, ammonium, calcium, or ferric hydroxides, and such organic bases as isopropylamine, trimethylamine, 2-ethylamino ethanol, histidine, and procaine.

Compositions of the invention are typically formulated as injectables, either as liquid solutions or suspensions; solid forms suitable for solution in, or suspension in, liquid prior to injection may also be prepared. The preparation may also be emulsified. The active immunogenic ingredient is often mixed with excipients, which are pharmaceutically acceptable and compatible with the active ingredient. Suitable excipients are, e.g., water, saline, dextrose, glycerol, ethanol, or the like and combinations thereof. In addition, if desired, the vaccine may contain minor amounts of auxiliary substances such as wetting or emulsifying agents, pH buffering agents, or adjuvants, which enhance the effectiveness of the vaccine. The vaccines are conventionally administered parenterally, by injection, for example, either subcutaneously or intramuscularly.

Additional formulations which are suitable for other modes of administration include suppositories and, in some cases, oral formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain 10%-95% of active ingredient, preferably 25-70%.

Compositions may also be administered through transdermal routes utilizing jet injectors, microneedles, electroporation, sonoporation, microencapsulation, polymers or liposomes, transmucosal routes and intranasal routes using nebulizers, aerosols and nasal sprays. Microencapsulation using natural or synthetic polymers such as starch, alginate and chitosan, D-poly L-lactate (PLA), D-poly DL-lactic-coglycolic microspheres, polycaprolactones, polyorthoesters, polyanhydrides and polyphosphazenes polyphosphatazanes are useful for both transdermal and transmucosal administration. Polymeric complexes comprising synthetic poly-omithate, poly-lysine and poly-arginine or amphipathic peptides are useful for transdermal delivery systems. In addition, due to their amphipathic nature, liposomes are contemplated for transdermal, transmucosal and intranasal vaccine delivery systems. Common lipids used for vaccine delivery include N-(1)2,3-(dioleyl-dihydroxypropyl)-N,N,N,-trimethylammonium-methyl sulfate (DOTAP), dioleyloxy-propyl-trimethylammonium chloride DOTMA, dimystyloxypropyl-3-dimethyl-hydroxyethyl ammonium (DMRIE), dimethyldioctadecyl ammonium bromide (DDAB) and 9N(N′,N-dimethylaminoethane)carbamoyl)cholesterol (DC-Chol). The combination of helper lipids and liposomes will enhance up-take of the liposomes through the skin. These helper lipids include dioleoyl phosphatidylethanolamine (DOPE), dilauroylphosphatidylethanolainine (DLPE), dimyristoyl phosphatidylethanolamine (DMPE), dipalmitoylphosphatidylethanolamine (DPPE). In addition, triterpenoid glycosides or saponins derived from the Chilean soap tree bark (Quillaja saponaria) and chitosan (deacetylated chitan) have been contemplated as useful adjuvants for intranasal and transmucosal vaccine delivery.

Formulations may be presented in unit-dose or multi-dose containers, for example, sealed ampules and vials and may be stored in a freeze-dried condition requiring only the addition of the sterile liquid carrier immediately prior to use. The dosage regimen will also, at least in part, be determined by the potency of the modality, the vaccine delivery employed, the need of the subject and be dependent on the judgment of the practitioner.

### Measuring all Immune Response

Immune responses according to the invention include the production of antibodies (a humoral response) to an antigenic polypeptide and the production and/or activation of CD4+ and CD8+ T cells (a cellular response) to the antigenic polypeptide encoded by the rAAV vector that has been administered, or both.

An “immunogenic dose” of a composition of the invention is one that generates, after administration, a detectable humoral (antibody) and/or cellular (T cell) immune response in comparison to the immune response detectable before administration or in comparison to a standard immune response before administration. The invention contemplates that the immune response resulting from the methods may be protective and/or therapeutic. In a preferred embodiment, the antibody and/or T cell immune response protects the individual from infection or persistence of the infectious agent causing a chronic infection. In this use, the precise dose depends on the patient's state of health and weight, the mode of administration, the nature of the formulation, etc., but generally ranges from about 1.0 μg to about 5000 μg per 70 kilogram patient, more commonly from about 10 to about 500 μg per 70 kg of body weight.

Humoral immune response may be measured by many well known methods, such as Single Radial Immunodiffussion Assay (SRID), Enzyme Immunoassay (EIA) and Hemagglutination Inhibition Assay (HAT). In particular, SRID utilizes a layer of a gel, such as agarose, containing the immunogen being tested. A well is cut in the gel and the serum being tested is placed in the well. Diffusion of the antibody out into the gel leads to the formation of a precipitation ring whose area is proportional to the concentration of the antibody in the serum being tested. EIA, also known as ELISA (Enzyme Linked Immunoassay), is used to determine total antibodies in the sample. The immunogen is adsorbed to the surface of a microtiter plate. The test serum is exposed to the plate followed by an enzyme linked inmunoglobulin, such as IgG. The enzyme activity adherent to the plate is quantified by any convenient means such as spectrophotometry and is proportional to the concentration of antibody directed against the immunogen present in the test sample. HAI utilizes the capability of an immunogen such as viral proteins to agglutinate chicken red blood cells (or the like). The assay detects neutralizing antibodies, i.e., those antibodies able to inhibit hemagglutination. Dilutions of the test serum are incubated with a standard concentration of immunogen, followed by the addition of the red blood cells. The presence of neutralizing antibodies will inhibit the agglutination of the red blood cells by the immunogen.

A “cellular immune response” is one mediated by T-lymphocytes and/or other white blood cells, including without limitation NK cells and macrophages. T lymphocytes of the present invention include T cells expressing alpha beta T cell receptor subunits or gamma delta receptor expressing T cells and may be either effector or suppressor T cells. Tests to measure cellular immune response include determination of delayed-type hypersensitivity or measuring the proliferative response of lymphocytes to target immunogen, or their production of cytokines like interferon gamma when stimulated with the target immunogen.

### Antigenic Polypeptides

An immune response against a bacterial antigenic polypeptide or antigen will provide immunity from bacterial infection or elicit an immune response to bacterial infection. Exemplary bacteria include Bacillus anthracis (anthrax), Borrelia burgdorferi (Lyme disease), Enterotoxigenic E. coli, Helicobacter pylori, Neisseria meningitis, Salmonella (non typhoidal), Salmonella typhi, Shiga toxin producing E. coli, Shigella spp., and Vibrio cholera, Bordetella pertussis, Chlamydia pneumoniae, Haemophilus influenzae B, Nontypeable Haemophilus influenzae, Moraxella catarrhalis, Mycobacterium tuberculosis, Mycoplasma pneumoniae, Pseudomonas aeruginosa, Smallpox, Staphylococcus aureus, Streptococci, Group A (GAS), Streptococci, Group B (GBS), and Tetanus., Chlamydia trachomatis, Neisseria gonorrhoeae, Treponemapallidum Leptospira spp., Staphylococcus saprophyticus and Uropathogenic E. coli.

An immune response against an viral antigenic polypeptide or antigen will provide immunity from viral infection or elicit an immune response to viral infection. For example, viral pathogens for which antigenic polypeptides may be obtained include: hepatitis A, hepatitis B, hepatitis C, hepatitis D, hepatitis E, hepatitis G, Epstein-Barr virus, cytomegalovirus, HIV-1, HIV-2, tuberculosis, SARS, influenza, Astrovirus, Campylobacter, Coxsackievirus, Echovirus, Norwalk virus, Poliovirus, and Rotavirus, Measles virus, Parainfluenza virus, Paramyxovirus, Respiratory syncytial virus, Rhinovirus, and Rubella virus, Human Papillomavirus, Cytomegalovirus, Epstein-Barr virus, Herpes simplex I, Herpes simplex II, Varicella zoster virus, Arbovirus, Dengue viruses, and Japanese encephalitis virus.

An immune response against a parasitic antigenic polypeptide or antigen is useful for treating parasitic infections or eliciting an immune response to a parasitic infection. Exemplary parasites include flagellates such as Giardia lamblia, Dientamoeba fragilis, Trichomonas hominis, Chilomastix mesnili, Enterornonas hominis, and Retortamonas intestinalis, Cestodes or helminths such as Taenia species, Hyrnenolepis nana, Hymenolepis diminuta, and Dihyllobothrium latum, Nematodes such as Enterobius vermicularis and Ascaris lumbricoides and Hookworms such as Trichuris trichiura, Strongyloides stercoralis. Additional exemplary parasites include Plasmodium falciparum, Plasmodium vivax, Plasmodium ovale, Plasmodium malariae, Schistosoma mansoni, Schistosoma haematobium, Schistosoma japonicum, Ascaris lumbricoides, Entamoeba histolytica, Enterobius vermicularis, Giardia lamblia, Mycobacterium leprae, Schistosoma spp., Taenia, Toxoplasma gondii and Trichomoniasis vaginalis.

An immune response against a antigenic polypeptide or antigen from a fungus is useful for treating fungal infections or eliciting an immune response to a fungal infection. Exemplary fungi include Aspergillus fumigatus, Blastomyces dermatitidis, Candida spp., Coccidioides iminitis, Cryptococcus neoformans, Histoplasma capsulatum and Paracoccidioides brasiliensis.

An immune response against cancer-associated or tumor-associated antigenic polypeptide are useful in treating cancers. Alternatively, tumor cells can also be transfected to express inhibitors of PD-1 signaling and an antigenic polypeptide. The transfected tumor cells are returned to the patient, which results in inhibition (e.g., local inhibition) of PD-1 signaling. Alternatively, gene therapy techniques of the invention can be used to target a tumor cell for transfection in vivo. Exemplary cancer-associated polypeptides may be obtained from leukemias, brain tumors (including meningiomas, glioblastoma multiforme, anaplastic astrocytomas, cerebellar astrocytomas, other high-grade or low-grade astrocytomas, brain stem gliomas, oligodendrogliomas, mixed gliomas, other gliomas, cerebral neuroblastomas, craniopharyngiomas, diencephalic gliomas, germinomas, medulloblastomas, ependymomas. choroid plexus tumors, pineal parenchymal tumors, gangliogliomas, neuroepithelial tumors, neuronal or mixed neuronal glial tumors), lung tumors (including small cell carcinomas, epidermoid carcinomas, adenocarcinomas, large cell carcinomas, carcinoid tumors, bronchial gland tumors, mesotheliomas, sarcomas or mixed tumors), prostate cancers (including adenocarcinomas, squamous cell carcinoma, transitional cell carcinoma, carcinoma of the prostatic utricle, or carcinosarcomas), breast cancers (including adenocarcinomas or carcinoid tumors), or gastric, intestinal, or colon cancers (including adenocarcinomas, invasive ductal carcinoma, infiltrating or invasive lobular carcinoma, medullary carcinoma, ductal carcinoma in situ, lobular carcinoma in situ, colloid carcinoma or Paget's disease of the nipple), skin cancer (including melanoma, squamous cell carcinoma, tumor progression of human skin keratinocytes, basal cell carcinoma, hemangiopericytoma and Karposi's sarcoma), lymphoma (including Hogkin's disease and non-Hodgkin's lymphoma), or sarcomas (including osteosarcoma, chondrosarcoma and fibrosarcoma).

### DETAILED DESCRIPTION

The following examples illustrate the invention wherein Example 1 demonstrates that rAAV vectors facilitate long-term expression of genes, Example 2 demonstrates that rAAV transduction induces PD-1 expression in CD8+ killer T lymphocyte, Example 3 describes construction of rAAV vectors expressing an antigenic polypeptide and an inhibitor of PD-1 signalling and Example 4 describes construction of rAAV vectors expressing a therapeutic protein and PD-L1. All of the U.S. Patents, U.S. Patent Applications International Patent Application and journal articles referred to herein are incorporated by reference herein in their entirety.

### Example 1

### AAV Vectors Facilitate Long-Term Expression of Genes

rAAV vectors are known to facilitate long-term expression of transgenes. A AAV vector human serotype 2 (2×10¹¹ drp) expressing the enhanced green fluorescent protein (eGFP) in phosphate buffered saline was delivered to the leg quadriceps muscle of Balb/c mice (Charles River) by intramuscular injection. An intense green fluorescence caused by eGFP expression was detected by imaging of the harvested leg muscle of the vaccinated mice 8 weeks after delivery of the rAAV vector to the leg muscle. However, eGFP expression was detectable for the entire life-span of the mouse.

The kinetics of PD-1 expression on eGFP-specific CD8+ T cells was analyzed on Days 7, 15, 21 and 56 post-injection. The T cells from spleen and liver of the vaccinated mice were analyzed. This analysis revealed high levels of PD-1 expression on eGFP-specific T cells from mice vaccinated with an Adenovirus vector or the rAAV vector. The expression of eGFP on T cells from mice treated with the Adenovirus vector decline over time, but eGFP expression increased or remained high on T cells from the rAAV treated mice.

As eGFP expression was detectable for the entire-life span of the mice, it was of interest to determine if T lymphocytes in these animals are primed to eliminate the rAAV transduced muscle cells. Lymphocytes from the spleen of mice immunized with the rAAV-eGFP vector were harvested 8 weeks after immunization. The lymphocytes were co-stained with fluorophore-labeled antibodies against CD8 (Becton Dickinson, Franklin Lakes, N.J.), which is a marker for the cytotoxic or killer subset of T cell lymphocytes, and an MHC class I tetramer that very specifically recognized CD8+ T cells directed against eGFP₂₀₀₋₂₀₈. CD8 and MHC class I tetramer staining was analyzed by flow cytometry. MHC class I tetramer-positive CD8+ T cells were found in the spleen 8 weeks after injection of the rAAV-eGFP vector, and were detectable for the entire natural life-span of the animal. Thus, failure to prime eGFP-specific CD8+ killer T cells cannot account for the inability to clear muscle cells expressing this model protein.

### Example 2

### rAAV Transduction Induces PD-1 Expression in CD8+ Killer T Lymphocytes

To further analyze the CD8+ killer T lymphocytes harvested from rAAV-immunized mice, the cells were co-stained with an antibody specific for PD-1 (Biolegend, San Diego, Calif.) and the fluorophore-labeled eGFP tetramer. The expression of PD-1 and the MHC class 1 tetramer were analyzed using flow cytometry. Almost all (96%) CD8+ killer T cells tested were positive for the eGFP MHC tetramer and expression of the inhibitory molecule PD-1. (FIG. 1)

In addition, muscle tissue transduced with rAAV vectors expressing eGFP to examined for expression of the PD-1 ligand, PD-L1 (Biolegend, San Diego, Calif.). Muscle cells from the rAAV-eGFP transduced leg expressed PD-L1 at high levels, whereas the contralateral (opposite) muscle from the same animal, that was not treated with an AAV vector, did not stain with anti-PD-L1 antibodies. Therefore, it is likely that the PD-1 positive CD8+ T cells encountering the PD-L1 ligand in AAV-transduced muscle tissue would receive signals that block immune functions, and thus prevent elimination of muscle cells expressing the eGFP model protein.

### Example 3

### Construction of AAV Vectors Expressing a Antigen and an Inhibitor of PD-1 Signaling

Viral, microbial, or parasitic antigens delivered to a host cell by a recombinant AAV vector may be processed to elicit an immune response and so is potentially useful for vaccination. As described above transduction of muscle cells with AAV induced PD-L1 expression on the transduced cells and expression of PD-1 on T cells. Upregulation of PD-1 and PD-L1 may down regulate the desired immune response to the target antigen and undermine vaccination. Expression of an inhibitor of PD-L1 gene or protein expression in cells transduced with AAV vectors in conjunction with a viral or microbial antigen may enhance priming of desired immune responses for vaccination.

AAV vectors are engineered to encapsidate a DNA plasmid with two genes, one encoding an inhibitor of PD-L1 gene or protein expression such as an siRNA, ribozyme, or anti-sense RNA molecule specific for the PD-L1 gene (Genbank Accession No. BC113734; SEQ ID NO:1; table 1) and one expressing an antigen of interest for instance proteins of viruses like the hepatitis C virus, human immunodeficiency virus, parasites like those causing malaria, or microbial pathogens like mycobacterium tuberculosis. A standard DNA plasmid containing inverted terminal repeats (ITRs) from the AAV genome that promote encapsidation by AAV capsid proteins are used. Genetic elements in the AAV DNA genome that are normally Ranked by the ITRs are removed and replaced with (i) DNA encoding the vaccine protein and (ii) genetic elements like silencing RNA molecules, ribozymes, or anti-sense KNA that prevent or reduce production of human PD-L1 protein. Expression of the vaccine gene is regulated by a standard polymerase I or II (POL I or II) promoter or an Internal Ribosome Entry Site (IRES) that facilitates protein synthesis. Expression of the PD-1 signaling inhibitors is regulated by a Pol I or II promoter. Examples of promoters that may be used are the human elongation factor one alpha (EF-1α) promoter or chicken beta actin (CBA) promoter. Promoters that have the additional advantage of imparting tissue specific (for instance HP1 which is a liver-specific promoter) expression of the vaccine protein and/or PD-L1 gene silencing element would also be used where applicable.

One example of a genetic construction is a PD-L1 gene or protein silencing element regulated by a POL I or II promoter followed by a gene encoding the vaccine protein driven by a second strong promoter like EF-1α, CBA (or a tissue-specific promoter) or an TRES element (such as . e.g. the encephalomyocarditis or polio virus IRES).

The genetic element containing the vaccine antigen(s) from the virus, bacteria, or parasite of interest and PD-L1 silencing genetic elements flanked by the AAV ITRs are encapsidated by AAV capsid proteins in a packaging cell line that is standard in the field. AAV particles containing this genetic element are purified from the producer cell culture by standard techniques used to prepare vector for safe administration to humans. Particles are delivered to the target organ of interest by direct injection into the tissue (for instance muscle, brain or liver), or through intravenous infusion. Uptake of the vector by cells in the targeted tissue results in constitutive expression of the target vaccine antigen and the inhibitor of PD-L1 gene or protein expression would facilitate priming of a functional T lymphocyte immune response to the target antigen.

### Example 4

### Construction of rAAV Vectors Expressing a Therapeutic Protien and PD-L1

Cells transduced with a rAAV vector present two potential targets for killer T cells. AAV coat proteins that encapsidate the genetic payload are the first target. They are carried into transduced cells and could be processed into peptides and presented for killer T cell recognition. Therapeutic proteins encoded by the genetic payload of the rAAV vector are the second target. As these therapeutic proteins are produced in target cells, some are processed into peptides for presentation to killer T cells. Cytotoxic activity by the killer T cells that is directed against rAAV capsid proteins or the encoded therapeutic protein(s) could result in lysis of the transduced cell and loss of a therapeutic effect.

Killer T cells directed against either target (i.e. the AAV capsid or therapeutic protein) express the PD-1 inhibitory receptor that will blunt or eliminate cytotoxic activity if it engages the PD-L1 receptor on a rAAV-transduced cell. PD-L1 expression on target cells is not necessarily constitutive (i.e. on all the time) and is thought to be up- or down- regulated by cytokines in the environment. Transient loss of the PD-L1 ligand on transduced cells would render them susceptible to attack by PD-1 positive T cells.

rAAV vectors are generated that constitutively express PD-L1 ligand on target cells are useful for gene therapy applications. rAAV vectors are engineered to encapsidate a DNA plasmid with two genes, one encoding a therapeutic protein designed to correct genetic error like such as, e.g. cystic fibrosis (CFTR) or hemophilia (Factor IX), and one expressing the human PD-L1 ligand. A standard DNA plasmid containing inverted terminal repeats (ITRs) from the AAV genome that promote encapsidation by rAAV particles are used. Genetic elements in the rAAV DNA genome that are normally flanked by the ITRs are removed and replaced with DNA encoding the therapeutic protein and human PD-L1. Expression of the therapeutic gene is regulated by a standard polymerase I or II (POL I or II) promoter or an Internal Ribosome Entry Site (IRES) that facilitates protein synthesis. Expression of the PD-L1 gene is regulated by a separate IRES or Pol I or II promoter. Where constitutive expression of PD-L1 is required, an IRES or polymerase promoter that is not susceptible to down regulation by cytokines is used. Examples of promoters that may be used are the human elongation factor one alpha (EF-1α) promoter or chicken beta actin (CBA) promoter. Promoters that have the additional advantage of imparting tissue specific (for instance HP1 which is a liver-specific promoter) expression of the therapeutic protein would also be used where applicable.

One example of a genetic construction is a therapeutic gene driven by a strong promoter like EF-1α, CBA (or a tissue-specific promoter) followed by a viral IRES element (such as. e.g. the encephalomyocarditis or polio virus IRES) driving expression of the PD-L1 gene.

The PD-L1 gene may also contain other standard elements including a signal sequence for co-translational synthesis in the endoplasmic reticulum and a transmembrane domain for targeting to the plasma membrane of the rAAV-transduced cell.

The genetic element containing the therapeutic and PD-L1 genes flanked by the rAAV ITRs are encapsidated by rAAV capsid proteins in a packaging cell line that is standard in the field. rAAV particles containing this genetic element are purified from the producer cell culture by standard techniques used to prepare vector for safe administration to humans. Particles are delivered to the target organ of interest by direct injection into the tissue (for instance muscle, brain or liver), or through intravenous infusion. Uptake of the vector by cells in the targeted tissue results in constitutive expression of the therapeutic protein and PD-L1 that would impart protection from immune recognition of the therapeutic protein by PD-1 positive killer T cells.

Alternatively, genes encoding the therapeutic protein and PD-L1 are contained in separate rAAV vectors that are co-delivered to the target tissue. This approach would be advantageous in settings where the therapeutic gene is near or at the maximum size that can be encapsidated in rAAV particles. The vector containing the PD-L1 gene would have the same genetic elements as those described above, with a promoter or IRES element that is not susceptible to regulation by factors in the microenvironment such as cytokines. This approach makes the reasonable assumption that at least a subset of target cells would be transduced by both vectors so that they both produce the therapeutic protein and PD-L1 required for protection from killer T cell attack.

What is claimed:

1. A method of modulating an immune response in a mammal treated with a recombinant AAV vector comprising administering to said mammal a recombinant AAV vector comprising a first nucleic acid encoding an antigenic or therapeutic polypeptide and administering a second nucleic acid encoding a modulator of PD-1 signaling.

2. The method of claim 1, wherein the administration of the recombinant AAV vector comprising a first nucleic acid and administration of the second nucleic acid are concurrent.

3. The method of claim 1, wherein administration of the recombinant AAV vector and administration of the second nucleic acid a is sequential.

4. The method of claim 1, wherein a single recombinant AAV vector comprises the first and second nucleic acids.

5. The method of claim 1, wherein a first recombinant AAV vector comprises the first nucleic acid and a second recombinant AAV vector comprises the second nucleic acid.

6. The method of claim 1, wherein the first nucleic acid encodes an antigenic polypeptide and the second nucleic acid encodes an inhibitor of PD-1 signaling.

7. The method of claim 6, wherein the second nucleic acid is administered in an amount effective to inhibit PD-1 signaling and the first nucleic acid is administered in an amount effective to elicit an immune response in said mammal to said antigenic polypeptide.

8. The method of claim 6, wherein the antigenic polypeptide is a polypeptide from a microbe and the immune response is protective.

9. The method of claim 6, wherein the antigenic polypeptide is a cancer-associated polypeptide and the immune response is therapeutic.

10. The method of claim 6 wherein the immune response to said antigenic polypeptide is enhanced compared to the immune response in the absence of inhibition of PD-1 signaling.

11. The method of claim 1 or 6, wherein the inhibitor of PD-1 signaling decreases expression of PD-1 or decreases the activity of PD-1.

12. The method of claim 1 or 6, wherein the inhibitor of PD-1 signaling decreases expression of PD-L1 or decreases the activity of PD-L1.

13. The method of claim 1 or 6, wherein the inhibitor of PD-1 signaling inhibits the interaction of PD-L1 and PD-1.

14. The method of claim 1 or 6, wherein the inhibitor of PD-1 signaling is selected from the group consisting of siRNA oligonucleotides, antisense oligonucleotides, soluble PD-1 fragments, antibodies or fragment thereof.

15. The method of claim 1, wherein the first nucleic acid encodes a therapeutic polypeptide and the second nucleic acid encodes an enhancer of PD-1 signaling.

16. The method of claim 15, wherein the enhancer is a PD-L1 polypeptide.

17. The method of claim 15, wherein the enhancer increases PD-L1 expression.

18. The method of claim 15, wherein the enhancer is a transcriptional regulatory element targeted to upregulate endogenous PD-L1 expression.

19. The method of claim 15, wherein the second nucleic acid is administered in an amount effective to suppress the mammal's immune response to the therapeutic polypeptide.

20. The method of claim 15, wherein the level of antibodies to the therapeutic polypeptide is decreased relative to the level in the absence of enhanced PD-1 signaling.

21. The method of claim 15, wherein the expression of the therapeutic polypeptide is prolonged.

22. The method of claim 15, wherein the therapeutic efficacy of the therapeutic polypeptide is prolonged.

23. The method of claim 15, wherein the dose of the nucleic acid encoding the therapeutic polypeptide is reduced compared to the dose in the absence of enhanced PD-1 signaling.

24. A method of inhibiting an immune response to a polypeptide comprising administering a recombinant AAV vector encoding the polypeptide and PD-L1 wherein PD-L1 inhibits the immune response.

25. A recombinant AAV vector comprising a first nucleic acid encoding an antigenic or therapeutic polypeptide and a second nucleic acid encoding a modulator of PD-1 signaling.

26. A cell transduced with said recombinant AAV vector of claim 25.

27. A method of producing said recombinant AAV vector of claim 25 comprising the steps of
(a) introducing a recombinant AAV vector comprising a first nucleic acid encoding an antigenic or therapeutic polypeptide and a second nucleic acid encoding a modulator of PD-1 signaling into a host cell;
(b) introducing an AAV helper virus construct into the host cell;
(c) introducing a helper virus into the host cell; and
(d) culturing the host cell to produce recombinant AAV virions of the recombinant AAV vector.

28. A composition comprising said recombinant AAV vector of claim 25.

29. A composition comprising (a) a first recombinant AAV vector comprising a first nucleic acid encoding an antigenic or therapeutic polypeptide and (b) a second recombinant AAV vector comprising a second nucleic acid encoding a modulator of PD-1 signaling.
